(19) United States
(12) Patent Application Publication
(10) Pub. No.: US 2005/0220866 A1
(43) Pub. Date: Oct. 6, 2005

(54) Novel capsule formulations of etoposide for oral use

(75) Inventors: Chandrashekhar Kocherlakota, Hyderabad (IN); Sarveswara Rao Srirama Mandavilli, Hyderabad (IN); Sandara Rao Basava Kolli, Hyderabad (IN); Divakar Manepalli, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignees: DR. REDDY'S LABORATORIES LIMITED; DR. REDDY'S LABORATORIES, INC.

(21) Appl. No.: 10/817,306

(22) Filed: Apr. 2, 2004

(51) Int. Cl. A61K 31/7048; A61K 9/48

(52) U.S. Cl. 424/451; 514/27

(57) ABSTRACT

The present invention relates to self microemulsifying pharmaceutical compositions comprising Etoposide that are encapsulated. The composition comprises (i) a drug phase comprising Etoposide, and a solvent; (ii) a co-solvent and (iii) an emulsifying base comprising a lipid, a surfactant and a stabilizer

### FIELD OF THE INVENTION

The present invention relates to self microemulsifying compositions comprising Etoposide that are encapsulated. These compositions can be used in the treatment of neoplastic diseases. Non-limiting examples of such diseases are refractory testicular cancer and small cell lung cancer. The self-microemulsifying formulation of Etoposide is designed to improve dissolution and bioavailability of Etoposide when administered orally.

### BACKGROUND OF THE INVENTION

Etoposide, i.e. 4′-demethylepipodophillotoxin-9-(4,6-O-ethylidene-.beta.-D glucopyranoside), has been used in the treatment of lung cancer, malignant lymphoma, and testicular tumor. Etoposide has been administered at a dosage of 100 mg/day by an oral route to obtain the required therapeutic activity. The effective administration of Etoposide to a subject is complicated by its poor solubility and as well by its other physico chemical properties.

Although formulations of Etoposide are known, Etoposide is difficult to solubilize in water and therefore, it is difficult to prepare an oral dosage form that provides the desired release of Etoposide.

An Etoposide capsule is a known dosage form (Cancer, April 1975, Vol. 35, No. 4, 1142). The composition of the capsule is reported to contain 100 mg of Etoposide, 320 mg of Miglyol 812, 70 mg of beeswax, and 10 mg of soya-lecithin.

U.S. Pat. No. 4,713,246 discloses a pharmaceutical solution dosage composition of Etoposide, that may be encapsulated, which is stable and free of precipitate and is acidic after dilution with water for a period of time sufficient to permit oral administration of said pharmaceutical dosage composition. However such compositions preferably require that Etoposide be in a micronised form.

U.S. Pat. No. 4,734,284 describes an Etoposide preparation comprising a vial or capsule and, enclosed therein, an Etoposide solution composition containing Etoposide and a water-soluble cellulose ether derivative or polyvinylpyrrolidone. According to the examples given in that U.S. Patent however, the preparation contains only 5-8% of Etoposide. Consequently it results in an excessively large capsule size when intended for use as an encapsulated preparation.

U.S. Pat. No. 4,772,589 discloses a stable solution of Etoposide which comprises Etoposide and a pharmaceutically acceptable acid in 1-methyl-2-pyrrolidinone that may be used for parenteral administration or may be encapsulated in a capsule shell.

U.S. Pat. No. 5,993,858 describes a self-microemulsifying excipient formulation which includes an emulsion, including an oil, or other lipid material, a surfactant, and a hydrophilic co-surfactant.

U.S. Pat. No. 5,929,030 discloses microemulsion preconcentrates for water-insoluble pharmaceutically active materials.

However none of above mentioned patents describes a self microemulsifying formulation of Etoposide.

The marketed formulations of Etoposide are soft gelatin capsules and solubilized solution formulations of Etoposide for oral or parenteral administration. There are commercially available embodiments of Etoposide in the form of Soft gelatin shell capsules which exhibit a drastic decrease in dissolution in pH 4.5 USP-buffer on storage.

A suitable self-microemulsifying formulation of Etoposide that would enhance oral drug absorption is not described in the prior art.

### SUMMARY OF THE INVENTION

This invention relates to compositions of Etoposide in the form of selfmicroemulsifying compositions. These compositions have been found to aid in the delivery of Etoposide. In addition, the combination of carrier material viz—the self microemulsifying base, solvent and cosolvent have been found to improve the dissolution characteristics of the active Etoposide from the dosage form.

The invention provides a self-microemulsifying composition of Etoposide with improved dissolution and enhanced absorption without any significant decrease of dissolution of the composition on storage.

In another aspect of the invention, the self-microemulsifying composition of Etoposide is encapsulated in a pharmaceutically acceptable capsule.

In yet another aspect the invention provides self-microemulsifying pharmaceutical compositions for oral use comprising Etoposide ranging from 25 mg to 100 mg of Etoposide per unit dose.

In still another aspect the invention provides a self-microemulsifying composition of Etoposide comprising a drug phase, a Cosolvent and self-microemulsifying phase with a HLB value ranging between 10.0 and 20.0.

Yet another aspect of the invention is to provide a method of manufacturing a Self-microemulsifying composition of Etoposide.

### BRIEF DESCRIPTION OF THE DRAWINGS

FIG. 1 shows comparative dissolution profile of Etoposide capsules in

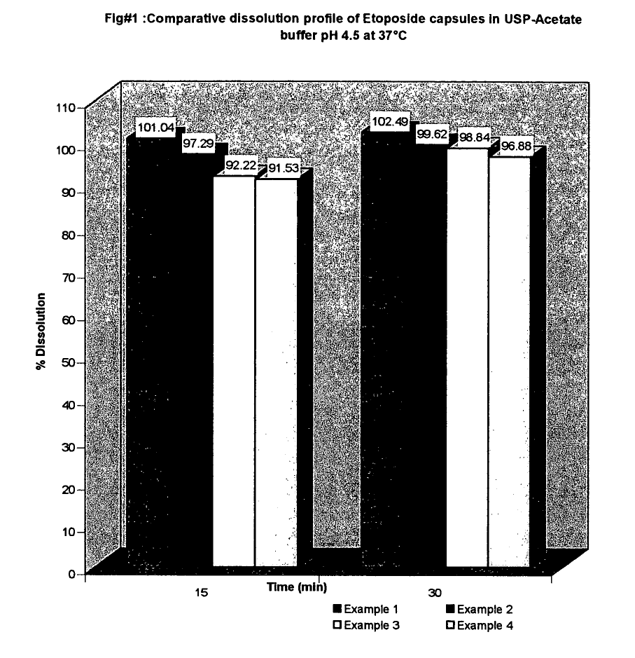

FIG. 2 shows comparative dissolution profile of Etoposide capsules in water at 37° C.

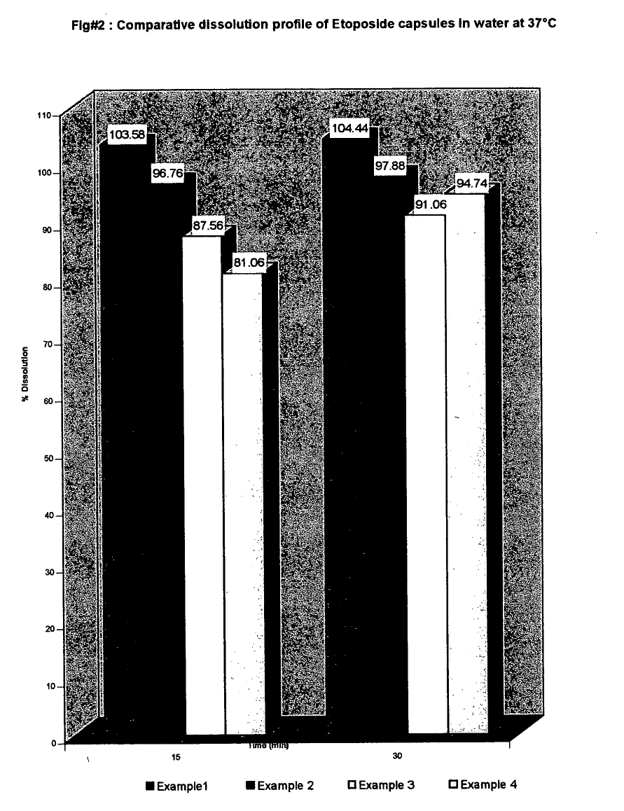

FIG. 3 shows comparative dissolution of a commercial sample of Etoposide and the composition of Example#1

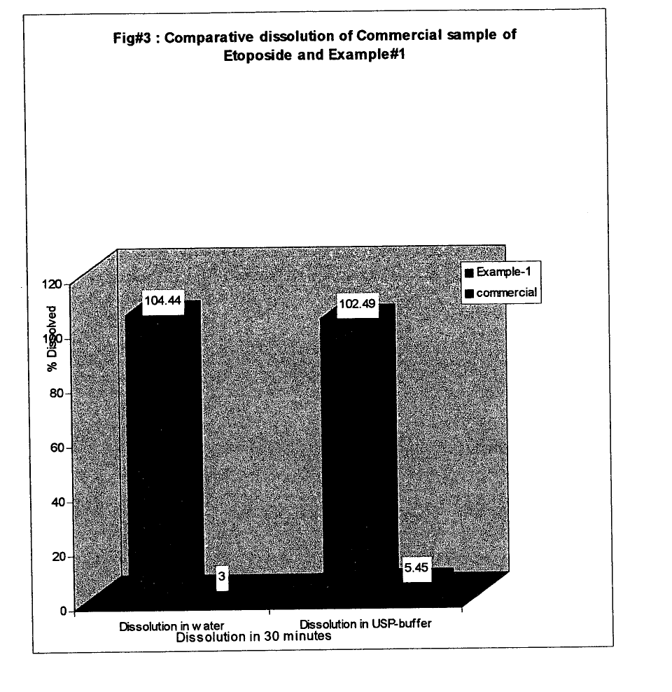

### DETAILED DESCRIPTION OF THE INVENTION

Unless defined otherwise, all technical and scientific terms used herein have the same meaning as commonly understood by one of ordinary skill in the art, to which this invention belongs. Methods and materials similar or equivalent to those described herein can be used in the practice or testing of the present invention.

Unless stated to the contrary, any use of the words such as “including,” “containing”, “comprising,” “having” and the like, means “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims.

For purposes of the present invention, the following terms are defined below.

A “compound” is a chemical substance that includes molecules of the same chemical structure.

“Pharmaceutically acceptable” means that which is useful in preparing a pharmaceutical composition that is generally non-toxic and is not biologically undesirable and includes that which is acceptable for veterinary use and/or human pharmaceutical use.

The term “composition” includes, but is not limited to, a solution, a suspension, an emulsion and/or mixtures thereof. The term composition is intended to encompass a product containing the specified ingredients in the specified amounts, as well as any product, which results, directly or indirectly, from combination of the specified ingredients in the specified amounts. A “composition” may contain a single compound or a mixture of compounds.

The term “pharmaceutical composition” is intended to encompass a product comprising the active ingredient(s), the other components and/or ingredients that are used to prepare the self-microemulsifying composition of Etoposide, pharmaceutically acceptable excipients if any, that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active ingredient, additional active ingredient(s), if any; the solvent(s), cosolvent(s), lipid(s), surfactant(s), and stabilizer(s) and pharmaceutically acceptable excipients, if any.

The term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for veterinary use as well as human pharmaceutical use. “A pharmaceutically acceptable excipient” as used in the specification and claims includes both one and more than one such excipient.

A “microemulsion” is formed when a self-microemulsifying composition of Etoposide is added to an aqueous solution in a ratio of 1:250 (1 part weight of self-microemulsifying composition of Etoposide to 250 parts by volume of aqueous solution) to 1:1000 (1 part weight of self-microemulsifying composition of Etoposide to 1000 parts by volume of aqueous solution). A microemulsion in an aqueous solution may be formed without the aid of any high shear agitation. The microemulsion formed appears transparent to translucent when observed visually.

Solvents and cosolvents-A co-solvent, in the scope of the present invention, is added in the selfmicroemulsifying composition of Etoposide to enhance the miscibility of solvent with selfmicroemulsifying base, to further aid in solubility of Etoposide, and/or to aid in formation of a microemulsion.

The term “Emulsifying base” means a composition comprising lipids, surfactants and stabilizers that has a HLB ranging between 10.0 and 20.0 and forms a micro emulsion.

The term “HLB value/hydrophilic lipophilic balance” is an empirical parameter commonly used to characterize the relative hydrophilicity and lipophilicity of non-ionic amphiphilic compounds. This is the hydrophilic-lipophilic balance (the “HLB” value). Surfactants with lower HLB values are more lipophilic, and have greater solubility in oils, whereas surfactants with higher HLB values are more hydrophilic, and have greater solubility in aqueous solutions. Using HLB values as a rough guide, hydrophilic surfactants are generally considered to be those compounds having an HLB value greater than about 10, as well as anionic, cationic, or zwitterionic compounds for which the HLB scale is not generally applicable. Similarly, lipophilic surfactants are compounds having an HLB value less than about 10.

It should be appreciated that the HLB value of a surfactant is merely a rough guide generally used to enable formulation of industrial, pharmaceutical and cosmetic emulsions. For many important surfactants, including several polyethoxylated surfactants, it has been reported that HLB values can differ by as much as about 8 HLB units, depending upon the empirical method chosen to determine the HLB value (Schott, J. Pharm. Sciences, 79(1), 87-88 (1990)). Likewise, for certain polypropylene oxide containing block copolymers (poloxamers, available commercially as PLURONIC.RTM. surfactants, BASF Corp.), the HLB values may not accurately reflect the true physical chemical nature of the compounds. Finally, commercial surfactant products are generally not pure compounds, but are often complex mixtures of compounds, and the HLB value reported for a particular compound may more accurately be characteristic of the commercial product of which the compound is a major component. Different commercial products having the same primary surfactant component can, and typically do, have different HLB values. In addition, a certain amount of lot-to-lot variability is expected even for a single commercial surfactant product. Keeping these inherent difficulties in mind, and using HLB values as a guide, one skilled in the art can readily identify surfactants having suitable hydrophilicity or lipophilicity for use in the present invention, as described herein.

### Surfactants

Surfactants can be used to provide any of several advantageous characteristics to the compositions of this invention, including: increased solubility of the active ingredient in the solid carrier; improved dissolution of the active ingredient; improved solubulization of the active ingredient upon dissolution; enhanced absorption and/or bioavailability of the active ingredient, and improved stability, both physical and chemical, of the active ingredient. The surfactant can be a single surfactant or a mixture of surfactants and can be ionic or non-ionic.

### Lipids

The term “lipid” means triglyceride derivatives of fatty acids, various pharmaceutically acceptable oils that contain glycerides, or glyceride derivatives of fatty acids.

Preferred triglycerides include vegetable oils, fish oils, animal fats, hydrogenated vegetable oils, partially hydrogenated vegetable oils, medium and long-chain triglycerides, and structured triglycerides. It should be appreciated that several commercial surfactant compositions contain small to moderate amounts of triglycerides, typically as a result of incomplete reaction of a triglyceride starting material in, for example, a transesterification reaction. Such commercial surfactant compositions, while nominally referred to as “surfactants”, may be suitable to provide all or part of the triglyceride component for the compositions of the present invention. Examples of commercial surfactant compositions containing triglycerides include some members of the surfactant families Gelucires (Gattefosse), Maisines (Gattefosse), and Imwitors (Huls). Specific examples of these compositions are: Gelucire 44/14 (saturated polyglycolized glycerides), Gelucire 50/13(saturated polyglycolized glycerides), Gelucire 53/10 (saturated polyglycolized glycerides), Gelucire 33/01 (semi-synthetic triglycerides of C₈-C₁₈saturated fatty acids), Gelucire 39/01 (semi-synthetic glycerides) and other Gelucires, such as 37/06, 43/01, 35/10, 37/02, 46/07, 48/09, 50/02, 62/05, etc.

### Stabilizers

The term “Stabilizer” means an agent or mixture incorporated into self-microemulsifying composition of Etoposide that would prevent degradation of Etoposide, and if used in the formulation, the capsule shell.

The term “Pharmaceutically acceptable capsule shell” includes both hard and soft capsules. A pharmaceutically acceptable capsule shell may also be a liquid-filled two-piece hard capsules.

Two-piece capsules consist of two parts—the body and a cap of slightly larger diameter which fits snugly over its open end. Such capsules are available in different sizes and colors and may be made of gelatin, hydroxypropylmethylcellulose or starch. Specific non limiting examples of capsules are two-piece gelatin capsules, two-piece capsules made from cellulosic raw materials that satisfy vegetarian and cultural needs, two-piece gelatin capsules that have been specially designed to be sealed for secure containment of liquids and semi-solids. Soft capsules are usually made of gelatin or starch and contain liquid preparations in a more flexible gelatin shell. Capsules that are coated may also be used.

When referring to a chemical reaction, the terms “treating”, “contacting” and “reacting” are used interchangeably herein and refer to adding or mixing two or more reagents under appropriate conditions to produce the indicated and/or the desired product. It should be appreciated that the reaction, which produces the indicated and/or the desired product, may not necessarily result directly from the combination of two reagents, which were initially added, i.e., there may be one or more intermediates which are produced in the mixture which ultimately leads to the formation of the indicated and/or the desired product.

The self-microemulsifying composition comprises Etoposide, a solvent, a co-solvent and a self-emulsifying phase.

The self-microemulsifying composition of this invention can readily be encapsulated in a pharmaceutically acceptable capsule shell such that the self-microemulsifying formulations of Etoposide would have improved dissolution in pH 4.5 USP buffer, purified water at 37 °±2° C., simulated gastric fluid, simulated intestinal fluid, and 0.1N HCl. In addition, the self-microemulsified compositions of Etoposide when encapsulated in a capsule shell do not show substantial reduction (Decrease in dissolution below 70%) in dissolution on storage.

Accordingly, the present invention provides self-microemulsifying formulations of Etoposide for oral use as described below:

TABLE 1
Composition of Self-microemulsifying formulation of Etoposide

| S. No. | Ingredient | % w/w |
|---|---|---|
| 1) | Drug Phase comprising | |
| (i) | Etoposide | 1% to 20% |
| (ii) | Solvent | 8% to 15% |
| 2) | Cosolvent | 5% to 25% |
| 3) | Emulsifying base comprising | Qs to 100% |
| (i) | Lipid | 5% to 20% |
| (ii) | Surfactant | 40% to 60% |
| (iii) | Stabilizer | 0.2% to 1.2% |

The Self-microemulsified formulation of Etoposide readily forms a microemulsion when diluted over a wide range (1:250 to 1:1000) with aqueous solutions such as Water, USP-Buffer, 0.1 N HCl, Simulated intestinal fluid (as described in USP) or Simulated gastric fluid (as described in USP).

The micro-emulsion obtained on addition of the self-microemulsying composition of Etoposide with any of the above listed aqueous solutions is translucent to clear and does not readily precipitate for a significant period of time (Significant period of time means: The microemulsion should not show greater than 10% precipitation within 30 minutes of its preparation).

The solvents used for dissolving Etoposide are not limited so long as they dissolve Etoposide by agitation, stirring or by heating or a combination thereof, in the range of 0.5 mg/mL to 250 mg/mL. The solvents may be liquid, semisolid or solid at room temperature and are pharmacologically and pharmaceutically acceptable. Non-limiting examples of such solvents are Dimethyl isosorbide, 1-methyl-2-pyrrolidone, N-methyl-pyrrolidone, and Dimethyl sulfoxide. A preferred solvent is 1-Methyl-2-pyrrolidone.

Non-limiting examples of a cosolvent that can be used in the self-microemulsifying composition of Etoposide include Diethyleneglycol-monoethylether, and Glycofurol. The preferred co-solvent is Diethyleneglycol-monoethylether.

The emulsifying base has an HLB value ranging between 10.0 and 20.0 and comprises a lipid, surfactant, and stabilizer.

The lipid used to prepare the emulsifying base is not limited so long as it forms a microemulsion in an aqueous system having a pH of 1.2 to 7.5, either alone or in presence of excipients known to emulsify, or solubilise, or combination of both, so that a microemulsion can be obtained.

The lipid may be liquid, semisolid or solid at room temperature and may solubilise or dissolve Etoposide on heating, stirring or agitation or a combination thereof.

Preferred lipids have a HLB between 10.0 and 15.0 and form a microemulsion in aqueous systems having a pH of 1.2 to 7.5, either alone or in association with surfactants. The lipids may have a bioavailability enhancing property Non-limiting examples of lipids to be used in self-microemulsifying base are Lauroyl macrogol-32-glycerides, Linoleoyl macrogol-6-glycerides, Caprylocaproyl macrogol-7 glycerides, Medium chain triglyceride oils, propylene glycol caprylate/caprate, propylene glycol derivatives of fatty acids, glyceryl esters of fatty acids, glycerol esters of fatty acids, and Fish lipid oils.

The purpose of the use of surfactant or surfactant mixtures is primarily meant to emulsify the drug phase containing Etoposide. The surfactant used in Self-microemulsifying base comprises either non-ionic surfactants, or anionic surfactants, or mixtures of non-ionic surfactants, or mixtures of anionic and non-ionic surfactants, such that the resulting HLB of the surfactant or the surfactant mixture is between 10.0 and 20.0.

The most preferred HLB range of the surfactant or the surfactant mixture to be used in self-microemulsifying composition of Etoposide ranges between 10.0 and 15.0.

Non-limiting examples of surfactants used in the Emulsifying base are Polysorbates, Sorbitan esters, polyethylene-propyleneglycol-copolymers, Polyoxyethylene castor oil derivatives, Caprylocaproyl Macrogol-8 glycerides, Propylene glycol laureate, Polyglyceryl-6-dioleate, Propylene glycol monocaprylates, Sodium lauryl sulphate, Docussate sodium, and bile salts.

The most preferred surfactants used in the Emulsifying base are Polysorbates, Sorbitan esters, polyethylene-propyleneglycol-copolymers, Polyoxyethylene castor oil derivatives, Caprylocaproyl Macrogol-8 glycerides, Propylene glycol laureate, Polyglyceryl-6-dioleate, and Propyleneglycol monocaprylates.

The stabilizers to be used in self-microemulsifying composition of Etoposide are not limited so long as they are compatible with Etoposide, the capsule shell, and do not hinder the self-microemulsifying property of the formulation. Non-limiting examples of stabilizers are antioxidants, carboxylic acids, and chelating agents that can be solid, semisolid or liquid at room temperature. Combinations of two or more of these substances could provide a synergistic effect and can stabilize Etoposide. Further the stabilizer could be suspended, solubilised or dissolved in the self-microemulsifying base either by heating, stirring or agitation or a combination thereof. Preferred stabilizers are derivatives of Tocopherol, Citric acid anhydrous, Acetic acid, Maleic acid, Succinic acid, Tartaric acid, Lactic acid, Sodium sulfite, Sodium meta bisulfite, Complexing agents, and Butylated Hydroxy toluene.

The most preferred stabilizers are mixtures of 27% w/v solution of Citric acid and Vitamin-E (Derived from natural source) in the ratio of 1:6 (Vitamin-E: Citric acid) weight by weight, added in the self-microemulsifying base.

The present invention also includes methods of preparation of self-microemulsifying formulations of Etoposide. A method of manufacturing a Self-microemulsifying composition of Etoposide with Etoposide ranging from 25 mg to 100 mg/unit dose comprises (i) dissolving Etoposide in Solvent, and cosolvent; (ii) combining the solution of (i) with the lipid, surfactant and stabilizer on, or both and (iii) filling into a pharmaceutically acceptable capsule shell.

The invention is further described by reference to the following examples which set forth in detail the preparation of compositions of the present invention. It will be apparent to those skilled in the art, that many modifications, both to materials, and methods, may be practiced without departing from the purpose and interest of this invention. The examples that follow are not intended to limit the scope of the invention as described hereinabove or as claimed below.

### EXAMPLE 1

| S. No. | Item | % w/w per capsule |
|---|---|---|
| 1. | Etoposide | 11.74 |
| 2. | N-methyl-pyrrolidone | 11.74 |
| 3. | Diethyleneglycol monoethyl ether | 23.47 |
| 4. | Polyoxyl 35 Castor Oil | 44.48 |
| 5. | Polysorbate-20 | 5.87 |
| 6. | Citric acid | 0.70 |
| 7. | Purified water | 1.88 |
| 8. | d-Alpha-Tocopherol Concentrate | 0.12 |
| | (derived from natural source) | |

### Method of Preparation

Step-1: Mix N-methyl-pyrrolidone and Diethyleneglycol monoethyl ether.

Step-2: Dissolve Etoposide in the above mixture.

Step-3: Add Polyoxyl 35 Castor Oil, d-Alpha-Tocopherol Concentrate (derived from natural source) and Polysorbate-20 to the drug solution obtained in Step-2 and stir until uniform.

Step-4: Dissolve Citric acid in purified water and to the solution obtained in Step-3 and stir until uniform.

Step-5: Fill the desired amount of solution obtained in Step-4 in starch, or gelatin capsules.

Step-6: Band seal the capsules if necessary.

### Example 2

| S. No. | Item | % w/w per capsule |
|---|---|---|
| 1. | Etoposide | 11.66 |
| 2. | N-methyl-pyrrolidone | 12.82 |
| 3. | Diethyleneglycol monoethyl ether | 13.99 |
| 4. | Medium chain triglyceride Oil | 0.58 |
| 5. | Polyoxyl 35 Castor | 37.30 |
| 6. | Polysorbate-20 | 8.16 |
| 7. | Caprylocaproyl macrogol-8-glycerides | 12.82 |
| 8. | Citric acid | 0.70 |
| 9. | Purified water | 1.86 |
| 10. | d-Alpha-Tocopherol Concentrate | 0.12 |
| | (derived from natural source) | |

### Method of Preparation

Step-1: Mix N-methyl-pyrrolidone, and Diethyleneglycol monoethyl ether.

Step-2: Dissolve Etoposide in the above mixture.

Step-3: Add Polyoxyl 35 Castor Oil, Medium chain triglyceride oil, d-Alpha-Tocopherol Concentrate (derived from natural source), Caprylocaproyl macrogol-8-glycerides and Polysorbate-20 to the drug solution obtained in Step-2 and stir until uniform.

Step-4: Dissolve Citric acid in purified water and to the solution obtained in Step-3 and stir until uniform.

Step-5: Fill the desired amount of solution obtained in Step-4 in starch, or gelatin capsules.

Step-6: Band seal the capsules if necessary.

### Example#3

| S. No. | Item | % w/w per capsule |
|---|---|---|
| 1. | Etoposide | 11.66 |
| 2. | N-methyl-pyrrolidone | 12.82 |
| 3. | Diethyleneglycol monoethyl ether | 12.82 |
| 4. | Medium chain triglyceride Oil | 0.58 |
| 5. | Polyoxyl 35 Castor Oil | 47.79 |
| 6. | Polysorbate-20 | 5.83 |
| 7. | Caprylocaproyl macrogol-8-glycerides | 5.83 |
| 8. | Citric acid | 0.70 |
| 9. | Purified water | 1.86 |
| 10. | d-Alpha-Tocopherol Concentrate | 0.12 |
| | (derived from natural source) | |

### Method of Preparation

Step-1: Mix N-methyl-pyrrolidone, and Diethyleneglycol monoethyl ether.

Step-2: Dissolve Etoposide in the above mixture.

Step-3: Add Polyoxyl 35 Castor Oil, Medium chain triglyceride oil, d-Alpha-Tocopherol Concentrate (derived from natural source), Caprylocaproyl macrogol-8-glycerides and Polysorbate-20 to the drug solution obtained in Step-2 and stir until uniform.

Step-4: Dissolve Citric acid in purified water and to the solution obtained in Step-3 and stir until uniform.

Step-5: Fill the desired amount of solution obtained in Step-4 in starch, or gelatin capsules.

Step-6: Band seal the capsules if necessary.

### Example 4

| S. No. | Item | % w/w per capsule |
|---|---|---|
| 1. | Etoposide | 11.66 |
| 2. | N-methyl-pyrrolidone | 12.82 |
| 3. | Diethyleneglycol monoethyl ether | 5.83 |
| 4. | Medium chain triglyceride Oil | 0.58 |
| 5. | Polyoxyl 35 Castor Oil | 54.78 |
| 6. | Polysorbate-20 | 5.83 |
| 7. | Caprylocaproyl macrogol-8-glycerides | 5.83 |
| 8. | Citric acid | 0.70 |
| 9. | Purified water | 1.86 |
| 10. | d-Alpha-Tocopherol Concentrate | 0.12 |
| | (derived from natural source) | |

### Method of Preparation

Step-1: Mix N-methyl-pyrrolidone, and Diethyleneglycol monoethyl ether.

Step-2: Dissolve Etoposide in the above mixture.

Step-3: Add Polyoxyl 35 Castor Oil, Medium chain triglyceride oil, d-Alpha-Tocopherol Concentrate (derived from natural source), Caprylocaproyl macrogol-8-glycerides and Polysorbate-20 to the drug solution obtained in Step-2 and stir until uniform.

Step-4: Dissolve Citric acid in purified water and to the solution obtained in Step-3 and stir until uniform.

Step-5: Fill the desired amount of solution obtained in Step-4 in starch, or gelatin capsules.

Step-6: Band seal the capsules if necessary.

The stability of self-microemulsified preparations of Etoposide when diluted in different ratios with the dissolution media is presented in Table#2 and Table#3

TABLE 2
1:250 dilution (1 part by weight of Etoposide self-microemulsifying
formulation is added to 250 parts by volume of Dissolution media)

| | Aqueous solutions/Dissolution media | | | | |
|---|---|---|---|---|---|
| | | | | | Simulated |
| | | Simulated | Water | USP-buffer | intestinal |
| Example | 0.1 N HCl | gastric fluid | pH between | for Etoposide | fluid |
| # | pH 1.2 | (USP) pH 1.2 | 5.50 to 6.50 | pH 4.50 | pH 7.20 |
| 1 | Clear and | Clear and | Clear and | Clear and | Clear and |
| | stable for | stable for | stable for | translucent | stable for |
| | 60 minutes | 60 minutes | 45 minutes | and stable | 45 minutes |
| | | | | for 45 | |
| | | | | minutes | |
| 2 | Clear and | Clear and | Clear and | Clear and | Clear and |
| | stable for | stable for | stable for | translucent | stable for |
| | 60 minutes | 60 minutes | 45 minutes | and stable | 45 minutes |
| | | | | for 45 | |
| | | | | minutes | |
| 3 | Clear and | Clear and | Clear and | Clear and | Clear and |
| | stable for | stable for | stable for | translucent | stable for |
| | 60 minutes | 60 minutes | 45 minutes | and stable | 45 minutes |
| | | | | for 45 | |
| | | | | minutes | |
| 4 | Clear and | Clear and | Clear and | Clear and | Clear and |
| | stable for | stable for | stable for | translucent | stable for |
| | 60 minutes | 60 minutes | 45 minutes | and stable | 45 minutes |
| | | | | for 45 | |
| | | | | minutes | |

| | |
|---|---|
| Acceptance criteria | The microemulsion shall not show greater than 10% |
| | precipitation of Etoposide when added to the above |
| | aqueous solution in 30 minutes of time after addition |

TABLE 3
1:1000 dilution (1 part by weight of Etoposide self-microemulsifying
formulation is added to 1000 parts by volume of Dissolution media)

| | Aqueous solutions/Dissolution media | | | | |
|---|---|---|---|---|---|
| | | Simulated | Water | USP-buffer | Simulated |
| Example | 0.1 N HCl | gastric fluid | pH between | for Etoposide | intestinal fluid |
| # | pH 1.2 | (USP) pH 1.2 | 5.50 to 6.50 | pH 4.50 | pH 7.20 |
| 1 | Clear solution | Clear solution | Clear solution | Translucent | Clear solution |
| | and stable for | and stable for | and stable for | and stable for | and stable for |
| | greater than | greater than | greater than | greater than | greater than |
| | 60 minutes | 60 minutes | 60 minutes | 45 minutes | 60 minutes |
| 2 | Clear solution | Clear solution | Clear solution | Translucent | Clear solution |
| | and stable for | and stable for | and stable for | and stable for | and stable for |
| | greater than | greater than | greater than | greater than | greater than |
| | 60 minutes | 60 minutes | 60 minutes | 45 minutes | 60 minutes |
| 3 | Clear solution | Clear solution | Clear solution | Translucent | Clear solution |
| | and stable for | and stable for | and stable for | and stable for | and stable for |
| | greater than | greater than | greater than | greater than | greater than |
| | 60 minutes | 60 minutes | 60 minutes | 45 minutes | 60 minutes |
| 4 | Clear solution | Clear solution | Clear solution | Translucent | Clear solution |
| | and stable for | and stable for | and stable for | and stable for | and stable for |
| | greater than | greater than | greater than | greater than | greater than |
| | 60 minutes | 60 minutes | 60 minutes | 45 minutes | 60 minutes |

| | |
|---|---|
| Acceptance criteria | The microemulsion shall not show greater than 10% |
| | precipitation of Etoposide when added to the above |
| | aqueous solution in 30 minutes of time after addition |

The self-microemulsifying compositions of Etoposide have the following advantages:
a) The Self-microemulsifying composition of Etoposide when encapsulated in a capsule shell made of Gelatin or Starch has a shelf life of at least two years.
b) The self-microemulsifying compositions of Etoposide comply with USP standards.

The following is the stability data of Example-1 at ICH conditions (International Conference on Harmonization) and analyzed using validated stability indicating method.

### Stability Data of Example-1 at Real Time Condition (25° C./60% RH)

| | | | Temperature (25° C./60% RH) | | | |
|---|---|---|---|---|---|---|
| S. No. | Parameter | USP Specification | Initial | 3-Months | 6-Months | 9-Months |
| 1 | Description | Pale yellow to Deep | Complies | Complies | Complies | Complies |
| | | yellow colored | | | | |
| | | Viscous solution. | | | | |
| 2 | Dissolution | Not less than 85.0% | 105.3-107.2 | 103.6-106.9 | 101.9-103.7 | 101.6-103.8 |
| 3 | Assay % | 90.0-110.0% per | 104.2 | 103.2 | 101.4 | 100.67 |
| | | capsule | | | | |
| 4 | Max. individual | Not more than 2.0% | 0.5% | 0.3% | 0.4% | 0.5% |
| | impurity | | | | | |
| 5 | Total impurities | Not more than 3.0% | 0.6% | 0.4% | 0.5% | 0.6% |

### Stability Data of Example-1 at Accelerated Time Condition (40° C./75% RH)

| | | | Temperature (40° C./75% RH) | | | |
|---|---|---|---|---|---|---|
| S. No. | Parameter | USP Specification | Initial | 1-Months | 3-Months | 6-Months |
| 1 | Description | Pale yellow to Deep | Complies | Complies | Complies | Complies |
| | | yellow colored | | | | |
| | | Viscous solution. | | | | |
| 2 | Dissolution as | Not less than 85.0% | 105.3-107.2 | 94.9-104.4 | 104.8-107.3 | 99.4-103.4 |
| | per USP | (Q) in 30 minutes | | | | |
| 3 | Assay % | 90.0-110.0% per | 104.2 | 103.5 | 103.8 | 101.27 |
| | | capsule | | | | |
| 4 | Max. individual | Not more than 2.0% | 0.5% | 0.4% | 0.3% | 0.4% |
| | impurity | | | | | |
| 5 | Total impurities | Not more than 3.0% | 0.6% | 0.8% | 0.4% | 0.4% |

Since the Selfmicroemulsifying composition of Etoposide is stable for 6 months at 40° C. and complies with USP specifications, a shelf-life of two years can be assigned to the product.
c) The percentage drug release of Self-microemulsifying composition of Etoposide does not change significantly during the shelf life of the dosage form.
d) The above described Self-microemulsifying compositions of Etoposide result in marked increase of dissolution which amounts to greater than 30% when compared to dissolution of Etoposide from available commercial embodiments.

e) The self-microemulsifying composition of Etoposide shall comply with the following dissolution specification through its shelf life.

| | % Release in | % Release in |
|---|---|---|
| Dissolution condition | 15 minutes | 30 minutes |
| Water at 37° C. and | Not less than 50% | Not less than 75% |
| at 50 rpm in USP- | | |
| Type-II apparatus | | |
| PH 4.5 USP- acetate | Not less than 50% | Not less than 85% |
| buffer at 37° C. | | |
| and at 50 rpm in USP- | | |
| Type-II apparatus | | |

When commercial soft gelatin capsule embodiment of Etoposide is analyzed for dissolution in water and pH 4.5 USP-buffer the dissolution did not exceed more than 10.0%. This is primarily due to the fact that the Commercial preparations of Etoposide do not form a stable microemulsion and cannot be diluted in aqueous solutions over the range of 1:250 and 1:1000.

The following is the dissolution data of Example-1, Example-2, Example-3 & Example-4 performed in pH 4.5 USP-Buffer at 37° C. using USP-Type-2 apparatus at 50 rpm:

| | Dissolution | Dissolution |
|---|---|---|
| Formulation | in 15 min | in 30 min |
| Example 1 | 101.08% | 102.49% |
| Example 2 | 97.29% | 99.62% |
| Example 3 | 92.22% | 98.84% |
| Example 4 | 91.53% | 96.88% |

The following is the dissolution data of Example-1, Eaxmple-2, Example-3 and Example-4 performed in water at 37° C. using USP-Type-2 apparatus at 50 rpm:

| | Dissolution | Dissolution |
|---|---|---|
| Formulation | in 15 min | in 30 min |
| Example 1 | 103.58% | 104.44% |
| Example 2 | 96.76% | 97.98% |
| Example 3 | 87.56% | 91.06% |
| Example 4 | 81.06% | 94.74% |

The following is the comparison of dissolution data of Example-1 with existing commercial Soft gelatin shell capsule formulation using pH 4.5 buffer as described in USP:

F) Because the self-microemulsifying composition of Etoposide in capsule dosage form forms a stable microemulsion upon dilution with Water, or 0.1 N HCl, or pH-4.5 USP-Buffer, or Simulated gastric fluid, or Simulated intestinal fluid, it would result in increase in bioavailability

1. A self-microemulsifying composition comprising Etoposide encapsulated in a pharmaceutically acceptable capsule shell.

2. The composition according to claim 1, comprising (i) a drug phase comprising Etoposide, and a solvent; (ii) a co-solvent and (iii) an emulsifying base comprising a lipid, a surfactant and a stabilizer.

3. The composition according to claim 1, comprising (i) a drug phase comprising Etoposide and a solvent selected from the group consisting of 1-methyl-2-pyrrolidone, N-methyl-pyrrolidone, dimethyl isosorbide and dimethyl sulfoxide or a mixture thereof, (ii) cosolvent selected from the group consisting of Diethyleneglycol-monoethylether, and Glycofurol or a mixture thereof and (iii) emulsifying base with a HLB value ranging between 10.0 and 20.0 comprising a lipid, surfactant, and stabilizer.

4. The composition according to claim 2, wherein Etoposide is in the range of from 1% to 20% weight/weight of the composition, the solvent is in the range of from 8% to 15% weight/weight of the composition, the Co-solvent is in the range of from 5% to 25% weight/weight of the composition and (iii) the amount of the emulsifying base is in the range from 40% to 86% weight, with respect to the total weight of the composition.

5. The composition according to claim 3, wherein Etoposide is in the range of from 1% to 20% weight/weight of the composition, the solvent is in the range of from 8% to 15% weight/weight of the composition, the Co-solvent is in the range of from 5% to 25% weight/weight of the composition and (iii) the amount of the emulsifying base is in the range from 40% to 86% weight, with respect to the total weight of the composition.

6. The composition according to claim 2, wherein the solvent is 1-methyl-2-pyrrolidine.

7. The composition according to claim 3, wherein the solvent is 1-methyl-2-pyrrolidine.

8. The composition according to claim 2, wherein the co-solvent is diethyleneglycol-mono-ethylether.

9. The composition according to claim 3, wherein the co-solvent is diethyleneglycol-mono-ethylether.

11. The composition according to claim 2, comprising (i) a drug phase comprising Etoposide and a solvent selected from N-methyl-pyrrolidone, or Dimethylisosorbide, (ii) cosolvent selected from Diethyleneglycol-monoethylether, and Glycofurol and (iii) emulsifying phase comprising a lipid(s) selected from the group consisting of Lauroyl macrogol-32-glycerides, Linoleoyl macrogol-6-glycerides, Caprylocaproyl macrogol-7 glycerides, Medium chain triglyceride oils, propylene glycol caprylate/caprate, propylene glycol derivatives of fatty acids, glyceryl esters of fatty acids, glycerol esters of fatty acids, and Fish lipid oils or a combination thereof; surfactant(s) selected from the group of Polysorbates, Sorbitan esters, polyethylene-propyleneglycol-copolymers, Polyoxyethylene castor oil derivatives, Caprylocaproyl Macrogol-8 glycerides, Propylene glycol laureate, Polyglyceryl-6-dioleate, Propylene glycol monocaprylates, Sodium lauryl sulphate, Docussate sodium, or bile salts or a combination thereof, and stabilizer(s) selected from the group consisting of antioxidants, carboxylic acids, and chelating agents or a combination thereof.

12. The composition of Etoposide according to claim 2 comprising Etoposide, N-methyl-pyrrolidone, Diethyleneglycolmonoethyether, medium chain triglyceride oils, Caprylocaproyl macrogol-7 glycerides and Lauroyl macrogol-32-glycerides, Polysorbates, Sorbitan esters, polyethylene-propyleneglycol-copolymers, Polyoxyethylene castor oil derivatives, Caprylocaproyl Macrogol-8 glycerides, Propylene glycol laureate, Polyglyceryl-6-dioleate, Propyleneglycol monocaprylates, propylene glycol derivatives of fatty acids, and glyceryl esters of fatty acids, Citric acid and Vitamin-E, and encapsulated in a pharmaceutically acceptable shell.

12. The composition according to claim 2 comprising etoposide, N-methyl-pyrrolidone, diethyleneglycol monoethyl ether, polyoxyl 35 Castor oil, polysorbate-20, citric acid and d-alpha tocopherol.

13. The self-microemulsifying composition according to claim 2, encapsulated in a pharmaceutically acceptable shell.
